(19) United States
(12) Patent Application Publication
(10) Pub. No.: US 2025/0318844 A1
(43) Pub. Date: Oct. 16, 2025

(54) Ultrasound Applicator With Laser

(71) Applicant: Profound Medical Inc., Mississauga (CA)

(72) Inventors: Christopher Driver, Oakville (CA); Yifan Yang, Maple (CA); Hooman Reza Zadeh Tabatabai, Richmond Hill (CA)

(21) Appl. No.: 19/063,892

(22) Filed: Feb. 26, 2025

Related U.S. Application Data
(60) Provisional application No. 63/632,703, filed on Apr. 11, 2024.

(51) Int. Cl. A61B 17/22; A61B 18/00; A61B 18/22; A61B 18/26

(52) U.S. Cl. CPC A61B 17/2202; A61B 18/26; A61B 2018/00577; A61B 2018/00994; A61B 2018/2211; A61B 2018/2261; A61B 2018/2283

(57) ABSTRACT

An ultrasound applicator includes a shaft having a proximal end, a tip, and a length measured between the proximal end and the tip with respect to an axis; a plurality of channels defined in the shaft and extending from the proximal end of the shaft along at least a portion of the length of the shaft, the plurality of channels including an ultrasound channel and an optical-energy channel; one or more ultrasound transducers disposed in the ultrasound channel, the ultrasound transducer(s) configured to produce ultrasound energy in a first direction; and one or more optical fibers disposed in the optical-energy channel, the optical fiber(s) having a first end configured to be optically coupled to a laser source and a second end configured to direct laser energy at a predetermined angle relative to the first direction.

### CROSS-REFERENCE TO RELATED APPLICATIONS

This application claims priority to U.S. Provisional Application No. 63/632,703, titled “Ultrasound Probe With Laser,” filed on Apr. 11, 2024, which is hereby incorporated by reference.

### TECHNICAL FIELD

This application relates generally to ultrasound therapy devices.

### BACKGROUND

Ultrasound therapy devices are used to treat a variety of conditions including benign prostatic hyperplasia (BHP), calcifications such as bladder stones, kidney stones, and tumors. For some conditions, an ultrasound applicator is inserted into the body to place the ultrasound transducers close to the target region. Obstructions, such as calcifications, may be located between the ultrasound therapy device and the target region that prevent at least some of the ultrasound energy from reaching the target region.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to an ultrasound applicator comprising a shaft having a proximal end, a tip, and a length measured between the proximal end and the tip with respect to an axis; a plurality of channels defined in the shaft and extending from the proximal end of the shaft along at least a portion of the length of the shaft, the plurality of channels including an ultrasound channel and an optical-energy channel; one or more ultrasound transducers disposed in the ultrasound channel, the ultrasound transducer(s) configured to produce ultrasound energy in a first direction; and one or more optical fibers disposed in the optical-energy channel, the optical fiber(s) having a first end configured to be optically coupled to a laser source and a second end configured to direct laser energy at a predetermined angle relative to the first direction.

In one or more embodiments, the predetermined angle has a range of about 120 degrees to about 240 degrees whereby the ultrasound energy is directed towards a first side of the shaft and the laser energy is directed towards a second side of the shaft. In one or more embodiments, the predetermined angle is about 180 degrees. In one or more embodiments, the ultrasound applicator further comprises an optical window defined in the shaft, the optical window aligned with the second end of the optical fiber(s) such that the laser energy passes through the optical window, the optical window optically transparent to one or more wavelengths of the laser energy.

In one or more embodiments, the predetermined angle is a first predetermined angle, and the ultrasound applicator further comprises a mirror configured to reflect the laser energy at a second predetermined angle relative to the first direction. In one or more embodiments, the mirror is disposed in the optical-energy channel. In one or more embodiments, the mirror is electromechanically actuated such that the second predetermined angle is variable.

In one or more embodiments, the predetermined angle has a range of about +60 degrees to about −60 degrees whereby the ultrasound energy and the laser energy are directed towards a same side of the shaft.

In one or more embodiments, the ultrasound applicator further comprises a laser disposed in the optical-energy channel and optically coupled to the first end of the optical fiber(s).

Another aspect of the invention is directed to a medical device comprising an ultrasound applicator; a laser optically coupled to the first end of the optical fiber(s); and a power supply electrically coupled to the ultrasound transducer(s).

Another aspect of the invention is directed to an ultrasound applicator comprising a shaft having a proximal end, a tip, and a length measured between the proximal end and the tip with respect to an axis; a plurality of channels defined in the shaft and extending from the proximal end of the shaft along at least a portion of the length of the shaft, the plurality of channels including an ultrasound channel and an optical-energy channel; one or more ultrasound transducers disposed in the ultrasound channel, the ultrasound transducer(s) configured to produce ultrasound energy in a first direction; and a laser disposed in the optical-energy channel, the laser configured to direct laser energy at a predetermined angle relative to the first direction.

In one or more embodiments, the predetermined angle has a range of about 120 degrees to about 240 degrees whereby the ultrasound energy is directed towards a first side of the shaft and the laser energy is directed towards a second side of the shaft. In one or more embodiments, the ultrasound applicator further comprises an optical window defined in the shaft, the optical window aligned with the laser such that the laser energy passes through the optical window, the optical window optically transparent to one or more wavelengths of the laser energy.

In one or more embodiments, the predetermined angle is a first predetermined angle, and the ultrasound applicator further comprises a mirror configured to reflect the laser energy at a second predetermined angle relative to the first direction. In one or more embodiments, the predetermined angle has a range of about +60 degrees to about −60 degrees whereby the ultrasound energy and the laser energy are directed towards a same side of the shaft.

Another aspect of the invention is directed to a method for performing thermal therapy, comprising positioning an ultrasound applicator relative to a target volume in a mammal; directing laser energy towards an obstruction located between the ultrasound applicator and the target volume, the laser energy emitted from one or more optical fibers and/or a laser disposed in an optical-energy channel defined in a shaft of the ultrasound applicator; reducing a size of the obstruction; and after the size of the obstruction is reduced, directing ultrasound energy towards the target volume, the ultrasound energy produced by one or more ultrasound transducers disposed in an ultrasound channel defined in the shaft of the ultrasound applicator.

In one or more embodiments, the obstruction comprises a calcification. In one or more embodiments, the laser energy is directed towards a first side of the shaft, the ultrasound energy is directed towards a second side of the shaft, and the method further comprises after the size of the obstruction is reduced, rotating the shaft to align the ultrasound transducer(s) with the target volume.

In one or more embodiments, the optical fiber(s) and/or the laser is/are configured to direct the laser energy in a first direction, and the method further comprises reflecting the laser energy, with a mirror, in a second direction towards the obstruction. In one or more embodiments, the method further comprises electromechanically pivoting the mirror to adjust the second direction.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a block diagram of a medical system according to one or more embodiments.

FIG. 2 is a simplified and partially transparent illustration of an ultrasound applicator according to one or more embodiments.

FIG. 3 is a simplified and partially transparent illustration of an ultrasound applicator according to one or more embodiments.

FIG. 4 is a simplified and partially transparent illustration of an ultrasound applicator according to one or more embodiments.

FIG. 5 is a simplified and partially transparent illustration of an ultrasound applicator according to one or more embodiments.

FIG. 6 is a simplified and partially transparent illustration of an ultrasound applicator according to one or more embodiments.

FIG. 7 is a simplified and partially transparent illustration of an ultrasound applicator according to one or more embodiments.

FIG. 8 is a simplified and partially transparent illustration of an ultrasound applicator according to one or more embodiments.

FIG. 9 is a simplified and partially transparent illustration of an ultrasound applicator according to one or more embodiments.

FIG. 10 is a flow chart of a method 1000 for performing thermal therapy according to one or more embodiments.

FIG. 11 is a simplified and partially transparent illustration of an ultrasound applicator to illustrate a second step of the method illustrated in FIG. 10.

FIG. 12 is a simplified and partially transparent illustration of an ultrasound applicator to illustrate a third step of the method illustrated in FIG. 10.

FIG. 13 is a simplified and partially transparent illustration of an ultrasound applicator to illustrate a fourth step of the method illustrated in FIG. 10.

### DETAILED DESCRIPTION

An ultrasound applicator includes an optical-energy channel in which an optical fiber is disposed. The optical fiber is optically coupled to a laser to transmit laser energy therefrom. The laser can be located in the optical-energy channel or can be external to the ultrasound applicator. The distal end of the optical fiber can be configured to direct the laser energy in a predetermined angular direction relative to the ultrasound transducer(s) and/or the ultrasound window in the ultrasound applicator. The shaft of the ultrasound applicator can be optically transparent to the laser energy. Additionally or alternatively, an optical window can be defined in the shaft to allow the laser energy to pass through. In some embodiments, the distal end of the optical fiber can be aligned with a mirror that can reflect the laser energy at a predetermined angle. The mirror can be electromechanically actuated in some embodiments to adjust the reflection angle.

In one or more embodiments, an ultrasound applicator includes an optical energy channel in which a laser is disposed. The laser is configured to direct the laser energy in a predetermined angular direction relative to the ultrasound transducer(s) and/or the ultrasound window in the ultrasound applicator. In some embodiments, the laser can be aligned with a mirror and/or an optical window.

Laser energy can be used in combination with ultrasound energy to treat a treatment volume, such as a calcification in a mammal such as a human. Treating the treatment volume with a combination of ultrasound energy and laser energy can be more efficient and/or effective than treating the treatment volume using ultrasound energy alone. In this embodiment, one or more laser fibers may be utilized in the device to expedite the treatment.

Additionally or alternatively, laser energy can be used to ablate and/or breakup an obstruction such as calcification that may prevent or limit ultrasound energy from passing to the treatment volume.

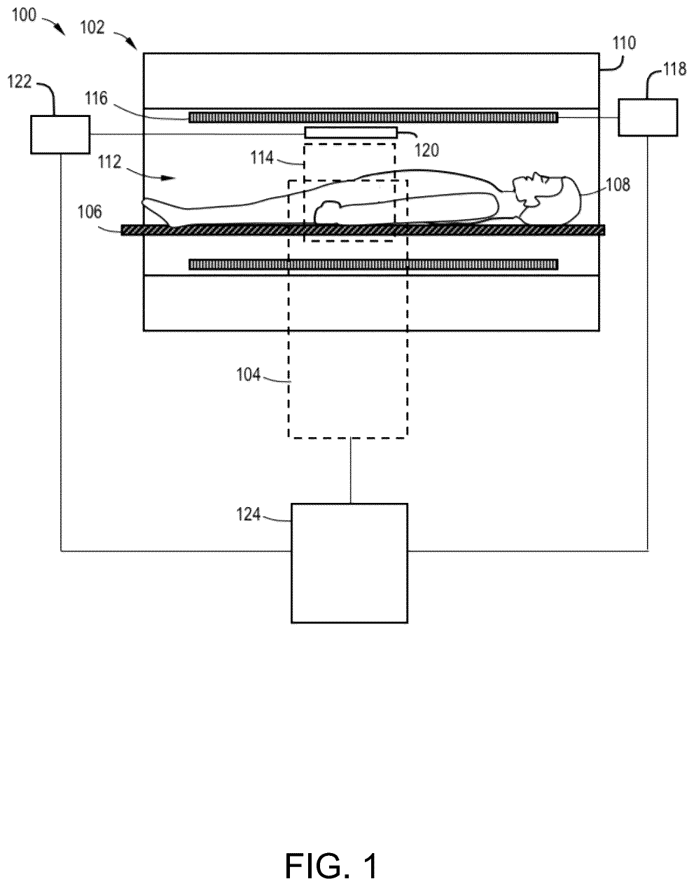

FIG. 1 is a diagram of a medical system 100 in which at least some of the apparatus, systems, and/or methods disclosed herein are employed, in accordance with at least some embodiments. The system 100 includes a patient support 106 (on which a patient 108 is shown), a magnetic resonance imaging (MRI) system 102 and an image-guided energy delivery system 104.

The magnetic resonance system 102 includes a magnet 110 disposed about an opening 112, an imaging zone 114 in which the magnetic field is strong and uniform enough to perform MRI, a set of magnetic field gradient coils 116 to change the magnetic field rapidly to enable the spatial coding of MRI signals, a magnetic field gradient coil power supply 118 that supplies current to the magnetic field gradient coils 116 and is controlled as a function of time, a transmit/receive coil 120 (also known as a “body” coil) to manipulate the orientations of magnetic spins within the imaging zone 114, a radio frequency transceiver 122 connected to the transmit/receive coil 120, and a computer 124, which performs tasks (by executing instructions and/or otherwise) to facilitate operation of the MRI system 102 and is coupled to the radio frequency transceiver 122, the magnetic field gradient coil power supply 118, and the image-guided energy delivery system 104. The image-guided energy delivery system 104 includes a therapeutic applicator, such as an ultrasound applicator, to perform image-guided therapy (e.g., thermal therapy) to treat a treatment volume in the patient 108.

The MRI computer 124 can include more than one computer in some embodiments, at least one of which can be dedicated to the MRI system 102. In at least some embodiments, the MRI computer 124 and/or one or more other computing devices (not shown) in and/or coupled to the system 100 may also perform one or more tasks (by executing instructions and/or otherwise) such as to control the driving or operating frequency of the ultrasound elements in the therapeutic applicator, such as at the center frequency (f₀) and/or at a higher harmonic (3f₀) of the center frequency.

One or more of the computers, including computer 124, can include a treatment plan for and/or program instructions for determining a treatment plan (e.g., in real time) for the patient 108 that includes the target treatment volume and the desired or minimal energy (e.g., thermal) dose for the target treatment volume. The treatment plan can also include the desired operating or driving frequency of the ultrasound elements, such as f₀ and/or 3f₀. The computer(s) can use images from the MRI system 102 to image guide the rotational position and insertion-retraction position of the therapeutic applicator. In some embodiments, one or more dedicated computers control the image-guided energy delivery system 104. Some or all of the foregoing computers can be in communication with one another (e.g., over a local area network, a wide area network, a cellular network, a WiFi network, or other network), for example through a software-controlled link to a communication network.

In some embodiments, the treatment plan includes a set of initial parameters for driving each ultrasound element such as its initial frequency, initial phase, and initial amplitude. These parameters can be updated in real time based on the measured temperature of the target volume, for example as determined by MR thermometry.

In other embodiments, the image-guided energy delivery system 104 can be guided using ultrasound produced by an ultrasound imaging device.

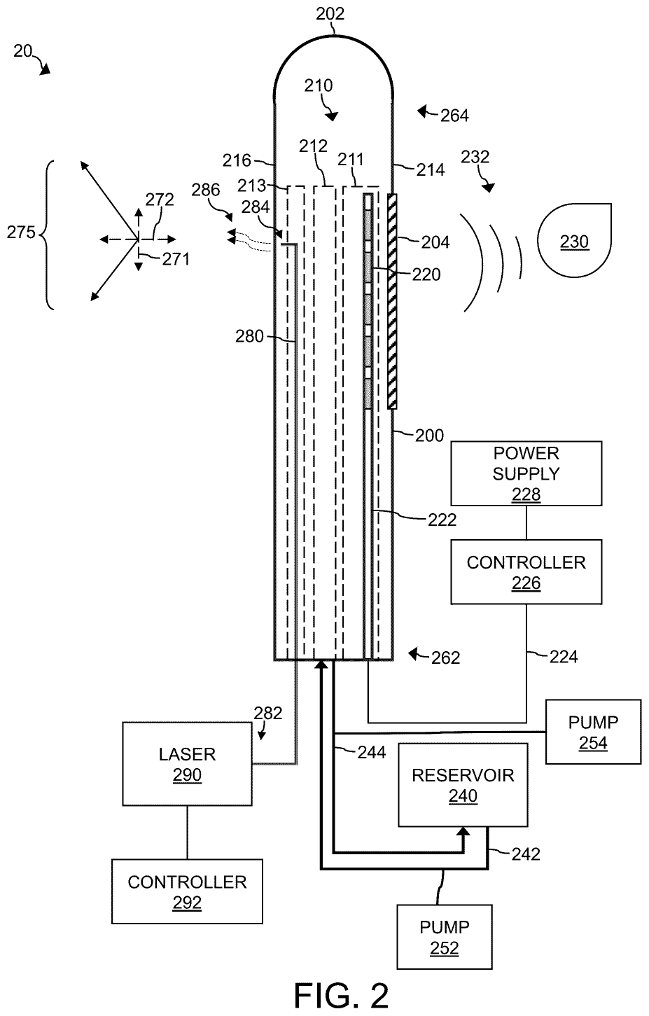

FIG. 2 is a simplified and partially transparent illustration of an ultrasound applicator 20 according to one or more embodiments. The ultrasound applicator 20 can be a therapeutic applicator for an image-guided energy delivery system 104 (FIG. 1). The ultrasound applicator 20 includes a shaft 200 attached to or including a tip 202. Multiple channels 210 can be defined in the shaft 200. Each channel 210 extends from a proximal end 262 towards or to a distal end 264 of the shaft 200. The shaft 200 and each channel 210 can extend parallel to a first axis 271, such that the respective lengths of the shaft 200 and each channel 210 can be measured with respect to the first axis 271.

The channel(s) 210 include an ultrasound channel 211 that is configured to receive or house one or more ultrasound transducers 220. The ultrasound transducer(s) 220 can comprise an array of ultrasound transducers, such as a linear array or a focused array of ultrasound transducers. The ultrasound transducer(s) 220 can be mounted on and/or electrically connected to an elongated circuit board 222. The elongated circuit board 222 can be electrically coupled (e.g., via wire(s) 224) to a controller 226 that can selectively provide electrical power, produced by a power supply 228, at a frequency, relative phase, and/or amplitude according to a treatment plan so as to treat a target volume 230 in a patient. The controller 226 and the power supply 228 can be combined in some embodiments. Ultrasound energy 232 produced by the ultrasound transducer(s) 220 is directed in a first direction towards a first side 214 of the shaft 200. In some embodiments, the ultrasound energy 232 can pass through an ultrasound window 204 defined or formed on the first side 214 of the shaft 200. The ultrasound energy 232 can be focused, geometrically and/or electronically, onto the target volume 230.

The channel(s) 210 can include a cooling channel 212 that is configured to receive cooling fluid (e.g., a cooling liquid such as water) that can be used to cool the ultrasound applicator 20 and/or the surrounding volume (e.g., surrounding tissue) during ultrasonic treatment. The cooling fluid can be provided from a cooling fluid reservoir 240. The cooling fluid can be recirculated between the cooling fluid reservoir 240 and the cooling channel 212. Cooler (e.g., room temperature) cooling fluid can flow from the cooling fluid reservoir 240 to the cooling channel 212 through an inlet line 242. After passing through at least a portion of the cooling channel 212 and receiving heat from the ultrasound applicator 20, warmer cooling fluid can flow from cooling channel 212 to the cooling fluid reservoir 240 through an outlet line 244. A pump 252 can be fluidly coupled to the inlet line 242 and/or a pump 254 can be fluidly coupled to the outlet line 244. Alternatively, a pump 252 or 254 can be fluidly coupled to the inlet line 242 and to the outlet line 244.

The channel(s) 210 include an optical energy channel 213. The optical energy channel 213 is configured to receive one or more optical fiber(s) 280 having a proximal end 282 that is configured to be optically coupled to a laser 290 (e.g., a laser source) to receive and transmit laser energy 286 produced by the laser 290. The laser 290 can be controlled by a controller 292. The controller 292 and the laser 290 can be combined in some embodiments.

A distal end 284 of the optical fiber 280 can be configured to direct laser energy 286 towards a second side 216 of the shaft 200. The first and second sides 214, 216 can be opposing sides of the shaft 200. Additionally or alternatively, the distal end 284 of the optical fiber 280 can be oriented and/or configured to direct laser energy 286 at a predetermined angle relative to the first direction of the ultrasound energy 232. For example, in FIG. 2, the ultrasound energy 232 is generally directed along or parallel to a second axis 272 that is orthogonal to the first axis 271. The width or diameter of the shaft 200 can be measured with respect to the second axis 272. In FIG. 2, the predetermined angle of the laser energy 286 is about 180 degrees relative to the first direction of the ultrasound energy 232 (e.g., where the ultrasound energy 232 is directed at or about 0 degrees and about parallel to the second axis 272). In one or more embodiments, a predetermined angle 275 of the laser energy 286 can be about 120 degrees to about 240 degrees relative to the first direction of the ultrasound energy 232 (e.g., where the ultrasound energy 232 is directed at or about 0 degrees and about parallel to the second axis 272), including about 150 degrees, about 180 degrees, about 210 degrees, and any values or ranges between any two of the foregoing values.

The shaft 200 can be optically transparent with respect to one or more wavelength(s) of light of the laser energy 286 produced by the laser 290. Alternatively, a hole and/or an optical window can be defined in the shaft 200 to allow the wavelength(s) of light of the laser energy produced by the laser 290 to pass through. The optical window can be optically transparent with respect to one or more wavelength(s) of light of the laser energy 286 produced by the laser 290.

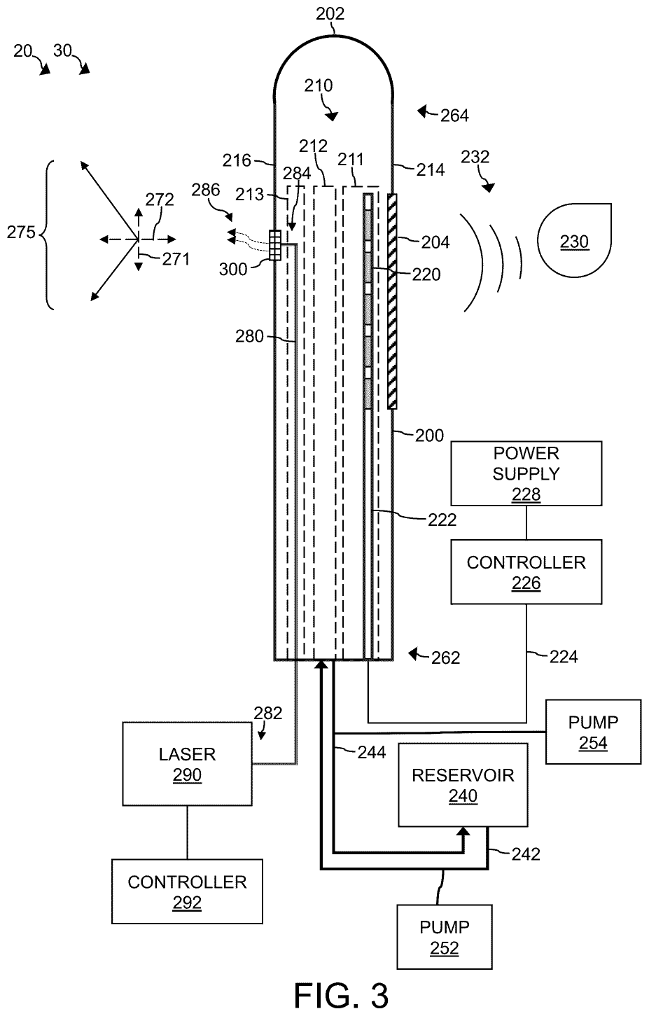

FIG. 3 is a simplified and partially transparent illustration of an ultrasound applicator 30 according to one or more embodiments. The ultrasound applicator 30 is the same as the ultrasound applicator 20 except that in the ultrasound applicator 30 the distal end 284 of the optical fiber 280 is aligned with an optical window 300 defined in the shaft 200. The optical window 300 is optically transparent to the wavelength(s) of light of the laser energy 286 produced by the laser 290. Thus, the optical window 300 allows the wavelength(s) of light of the laser energy 286 produced by the laser 290 to pass through to the shaft 200, for example to treat a target volume 230 and/or to fragment an obstruction, such as a calcification, between the ultrasound applicator 30 and the target volume 230.

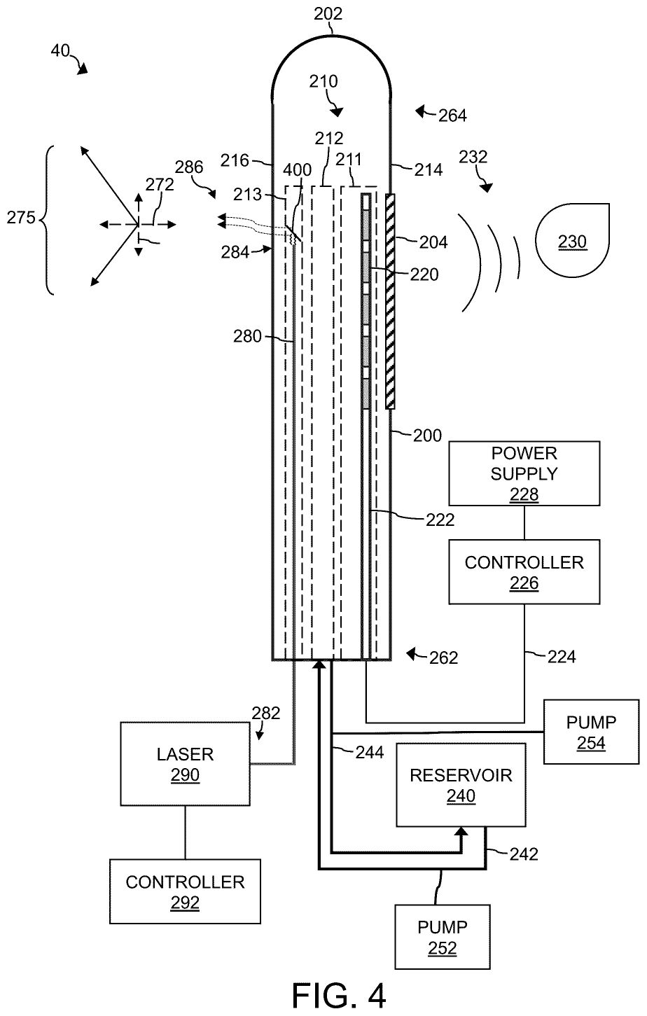

FIG. 4 is a simplified and partially transparent illustration of an ultrasound applicator 40 according to one or more embodiments. The ultrasound applicator 40 is the same as the ultrasound applicator 20 except that in the ultrasound applicator 40 a mirror 400 is disposed in the optical energy channel 213. The mirror 400 is aligned with the distal end 284 of the optical fiber 280 to reflect the laser energy 286 at a predetermined angle. The mirror 400 can be stationary or can be electromechanically actuated (e.g., via a controller in electrical communication with the mirror 400) to adjust the reflection angle of the laser energy 286.

The embodiments illustrated in FIGS. 3 and 4 can be combined. For example, a mirror 400 (FIG. 4) can reflect the laser energy 286 and the reflected laser energy 286 can pass through an optical window 300 (FIG. 3).

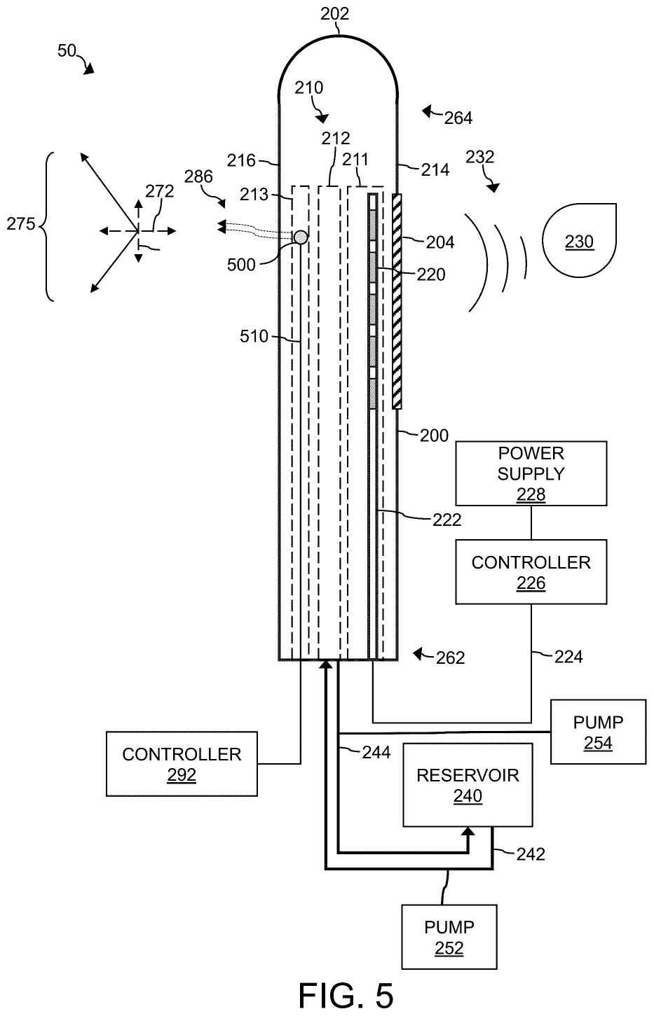

FIG. 5 is a simplified and partially transparent illustration of an ultrasound applicator 50 according to one or more embodiments. The ultrasound applicator 50 is the same as the ultrasound applicator 20 except that in the ultrasound applicator 50 a laser 500 is disposed in the optical energy channel 213 instead of optical fiber(s) 280. The laser 500 is configured to produce laser energy 286 in a predetermined angular direction relative to the ultrasound transducer(s) 220 and/or the ultrasound window 204. The laser 500 can be controlled by a controller 292 that is electrically and/or wirelessly coupled thereto. An electrical connection between the laser and the controller 292 can be implemented, for example, using one or more wire(s) or cable(s) 510.

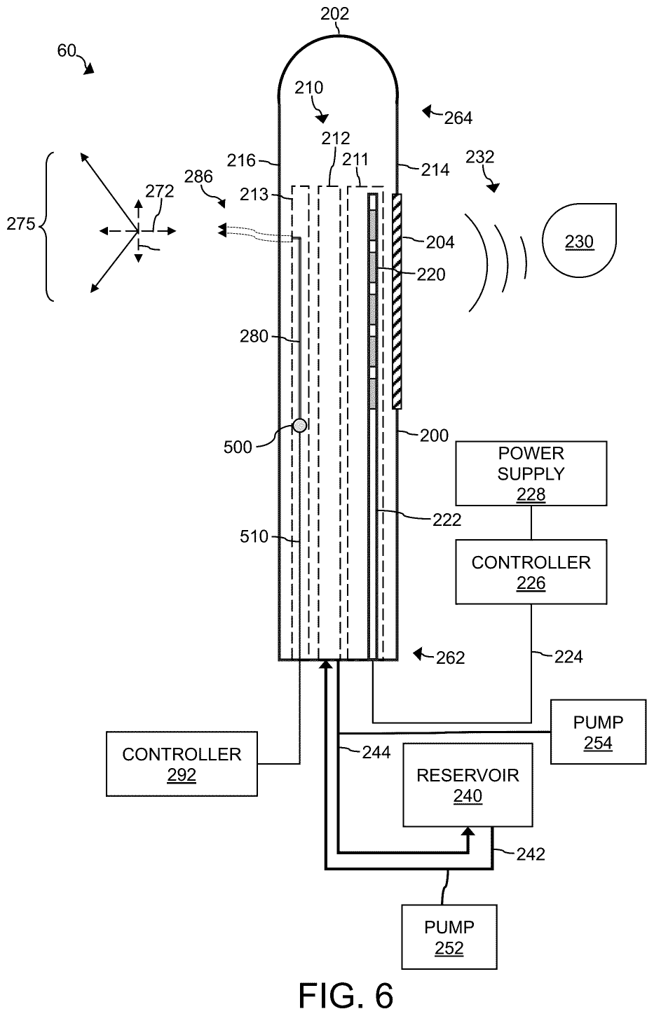

The embodiments illustrated in FIGS. 3, 4 and/or 5 can be combined. For example, a mirror 400 (FIG. 4) can reflect the laser energy 286 produced by the laser 500. Additionally or alternatively, the laser energy 286 produced by the laser 500 can pass through an optical window 300 (FIG. 3). Additionally or alternatively, a mirror 400 (FIG. 4) can reflect the laser energy 286 produced by the laser 500 and the reflected laser energy 286 can pass through an optical window 300 (FIG. 3). Additionally or alternatively, the laser 500 can be optically coupled to optical fiber(s) 280 (FIG. 2), for example in ultrasound applicator 60 illustrated in FIG. 6 according to one or more embodiments. The ultrasound applicator 60 is the same as the ultrasound applicator 50 except that the optical fiber(s) 280 are optically coupled to the laser 500.

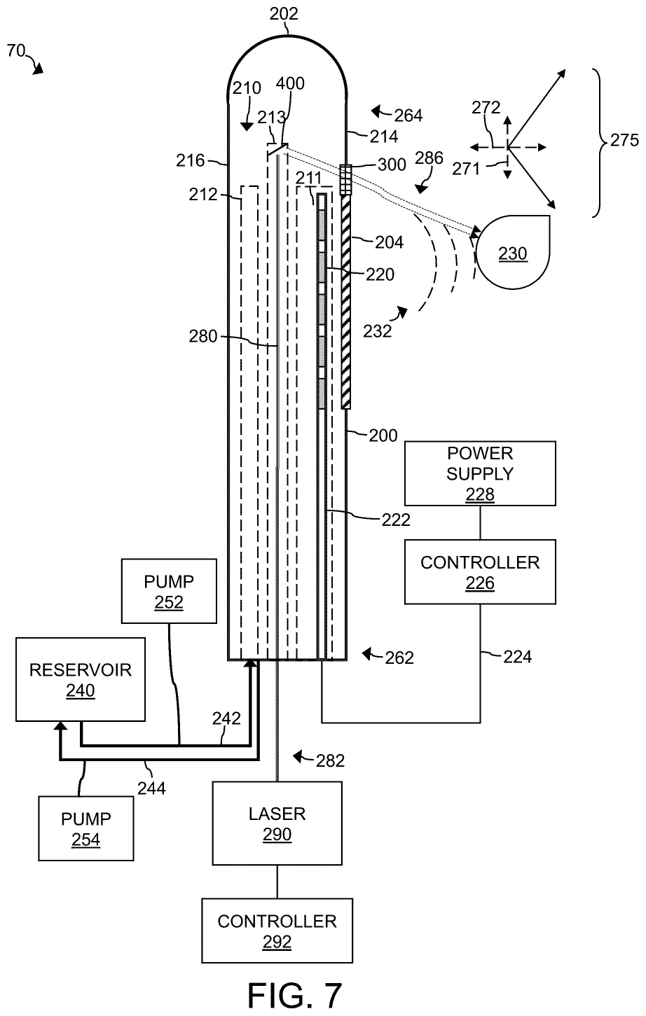

FIG. 7 is a simplified and partially transparent illustration of an ultrasound applicator 70 according to one or more embodiments. The ultrasound applicator 70 is the same as the ultrasound applicator 40 except that in the ultrasound applicator 70 the optical fiber 280 is configured to direct laser energy 286 towards the first side 214 of the shaft 200 in the same direction as the ultrasound energy 232. An optional mirror 400 can be used to direct the laser energy 286 towards the first side 214 of the shaft 200 such that the laser energy 286 is aligned with the ultrasound energy 232 at a predetermined distance from the first side 214 of the shaft 200, such as at a focal distance of the ultrasound energy 232. An optional optical window 300 through which the laser energy 286 can pass can be disposed on the first side 214 of the shaft 200.

The mirror 400 can reflect the laser energy 286 at a predetermined angle 275 of about +60 degrees to about −60 degrees relative to the first direction of the ultrasound energy 232 (e.g., where the ultrasound energy 232 is directed at or about 0 degrees and about parallel to the second axis 272), including about 150 degrees, about 180 degrees, about 210 degrees, and any values or ranges between any two of the foregoing values.

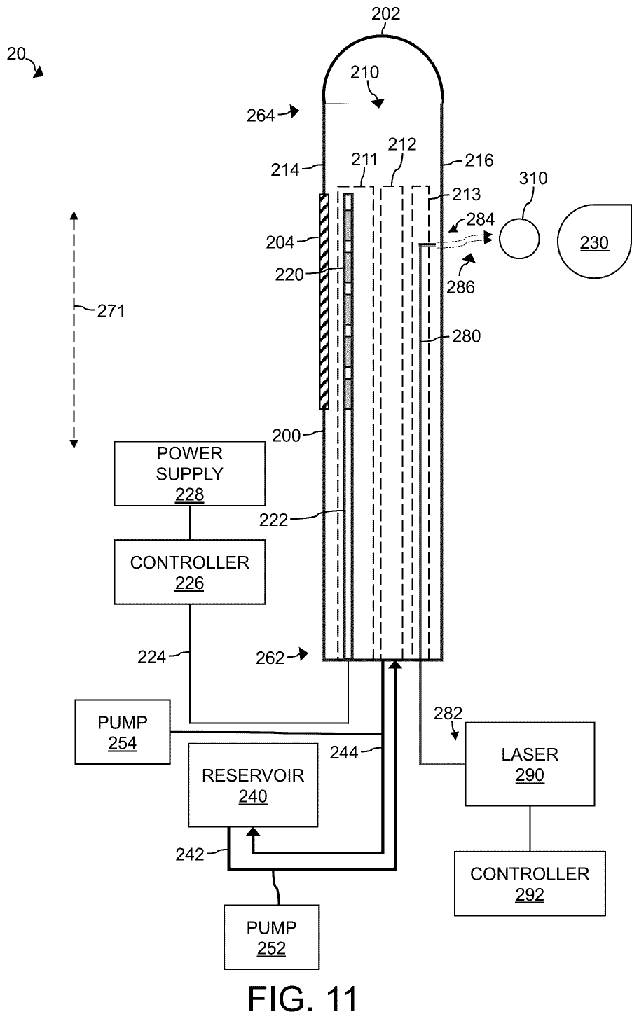

In some embodiments, laser energy 286 and ultrasound energy 232 can be produced simultaneously and/or in cycles to ablate a target volume 230. Additionally or alternatively, laser energy 286 can be used to break apart a calcification (e.g., obstruction 310 (FIGS. 11-13)) before the target volume 230 is ablated (e.g., using laser energy 286 and/or ultrasound energy 232). The laser energy 286 and the ultrasound energy 232 can be used without having to rotate the shaft 200.

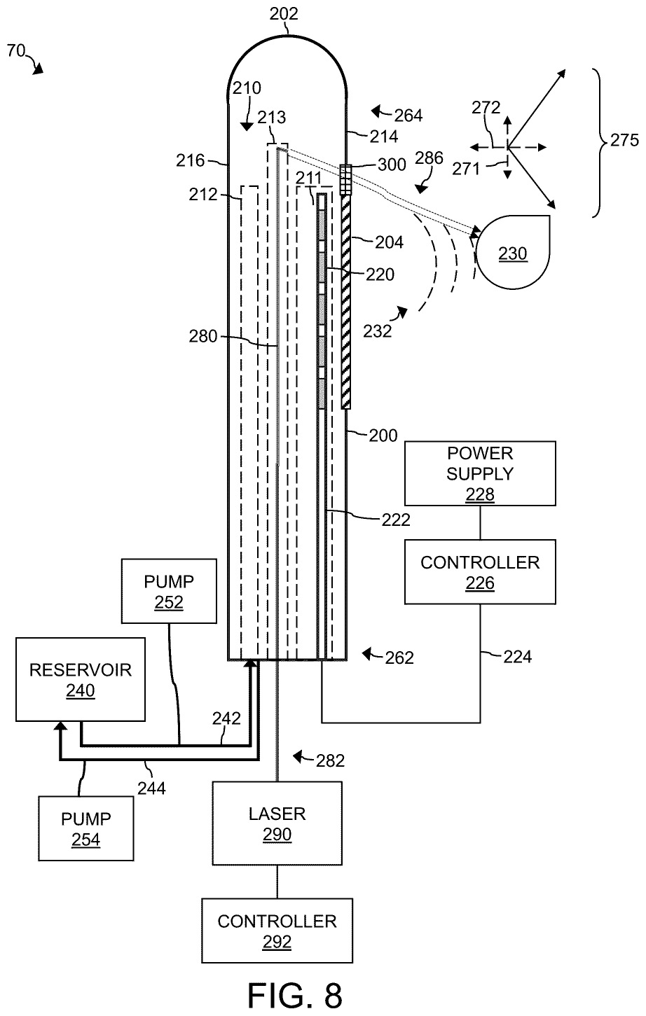

In some embodiments, the optical fiber(s) 280 can be bent or angled, instead of or in addition to including the optional mirror 400, so as to direct laser energy 286 towards the first side 214 of the shaft 200 and, optionally, in alignment with the ultrasound energy 232 at a predetermined distance from the first side 214 of the shaft 200, for example as shown in FIG. 8. The bent/angled optical fiber(s) 280 can emit the laser energy 286 at a predetermined angle 275 of about +60 degrees to about −60 degrees relative to the first direction of the ultrasound energy 232 (e.g., where the ultrasound energy 232 is directed at or about 0 degrees and about parallel to the second axis 272), including about 150 degrees, about 180 degrees, about 210 degrees, and any values or ranges between any two of the foregoing values.

In some embodiments, a laser 500 (FIGS. 5, 6) can be included instead of or in addition to the optical fiber(s) 280 and/or the laser 290.

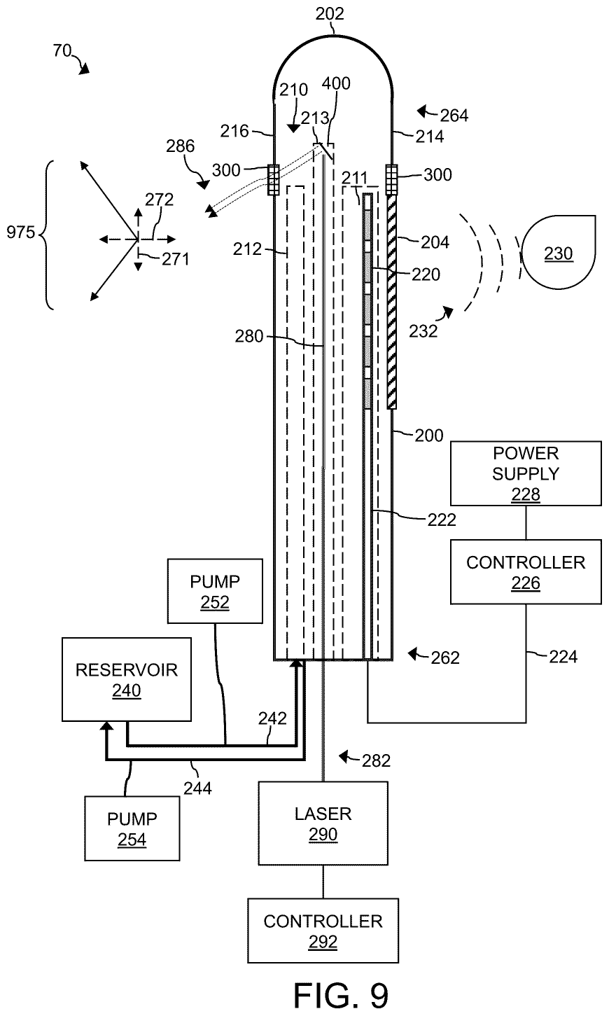

In some embodiments, the mirror 400 can be mechanically actuated to adjust a deflection angle of the laser energy 286. For example, the mirror 400 can be adjusted to have a first state or a first deflection angle 275 in which the laser energy 286 is aligned with the target volume 230, for example as shown in FIG. 7. Additionally or alternatively, the mirror 400 can be adjusted to have a second state or a second deflection angle 975 in which the laser energy 286 is directed towards the second side 216 of the shaft 200 (e.g., through a second optional optical window 300 on/in the second side 216 of the shaft 200), for example as shown in FIG. 9, for example to apply the laser energy 286 onto an obstruction. The second deflection angle 975 can be about 120 degrees to about 240 degrees relative to the first direction of the ultrasound energy 232 (e.g., where the ultrasound energy 232 is directed at or about 0 degrees and about parallel to the second axis 272), including about 150 degrees, about 180 degrees, about 210 degrees, and any values or ranges between any two of the foregoing values.

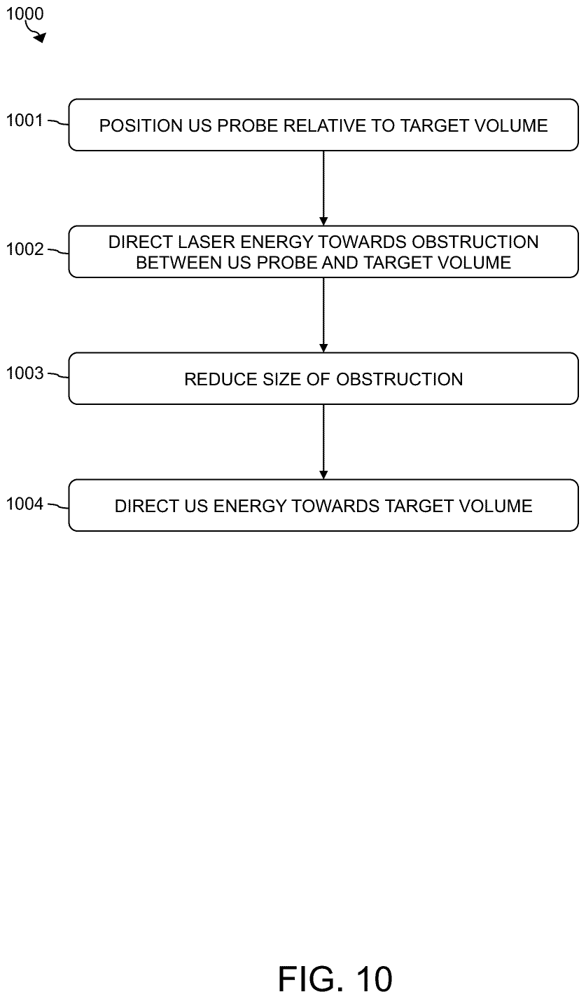

FIG. 10 is a flow chart of a method 1000 for performing thermal therapy according to one or more embodiments.

In step 1001, an ultrasound applicator is positioned relative to a target volume (e.g., a target volume 230). The target volume can represent a target location for thermal therapy. For example, the target volume can comprise a calcification (e.g., a stone such as a bladder stone or a kidney stone), a tumor, or another object or feature. The target volume can be located in a mammal such as a human. The ultrasound applicator can be positioned using imaging, such as ultrasound imaging, MRI, and/or other imaging. The ultrasound applicator can be the same as ultrasound applicator 20, 30, 40, 50, 60, or 70.

In step 1002, laser energy is directed towards an obstruction located between the ultrasound applicator and the target volume. The laser energy is emitted and/or produced from the ultrasound applicator. For example, the laser energy can be produced by a laser located in an optical energy (e.g., a laser) channel defined in the ultrasound applicator. Additionally or alternatively, the laser energy can be emitted from one or more optical fibers in the optical energy channel. The optical fiber(s) can be optically coupled to an internal laser in the optical energy channel or an external laser located externally from the optical energy channel and/or from the ultrasound applicator. The laser energy can be oriented at a predetermined angle and/or at a predetermined angular range relative to an axis along which the shaft of the ultrasound applicator extends. In some embodiments, a mirror can reflect the laser energy at the predetermined angle and/or at the predetermined angular range.

In some embodiments, the obstruction can be detected using any imaging used in the ultrasound position step 1001. Additionally or alternatively, the obstruction can be detected while thermal therapy is being applied to the target volume, for example using one or more ultrasound transducers in the ultrasound applicator. For example, some or all of the ultrasound energy directed to the target volume may not reach the target volume due to an obstruction, such as a calcification, located between the ultrasound applicator and the target volume and at least partially aligned with the ultrasound energy. Detection can occur automatically or manually (e.g., by a human), for example by determining that the temperature of the target volume (e.g., as measured by MRI thermometry) is not increasing at a slower rate than a target rate and/or than an expected temperate rate.

In some embodiments, after an obstruction 310 is detected and/or before applying the laser energy 286 in step 1002, an ultrasound applicator 20 can be rotated (e.g., by 180 degrees) to align the laser energy 286 with the obstruction 310, for example as shown in FIG. 11. The ultrasound applicator 20 shown in FIG. 11 is rotated by 180 degrees compared to the ultrasound applicator 20 shown in FIG. 2, such that the second side of the 216 of the shaft 200 is located closer to the obstruction 310 and to the target volume 300 compared to the first side 214 of the shaft 200. In FIG. 11, the ultrasound applicator 20 can be replaced with the ultrasound applicator 30, 40, 50, 60, or 70.

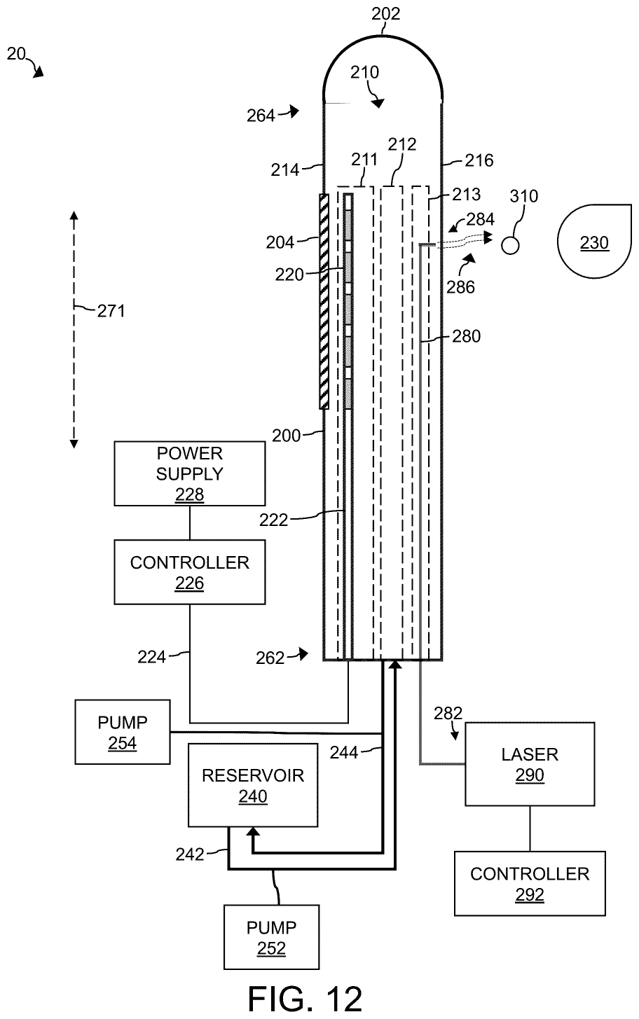

In step 1003, the size and/or volume of the obstruction 310 is/are reduced, for example as shown in FIG. 12. For example, the obstruction can be fragmented, broken, and/or disintegrated.

In step 1004, ultrasound energy is directed towards the target volume after the size and/or volume of the obstruction is/are reduced in step 1003. Some or all of the ultrasound energy can reach the target volume after the size and/or volume of the obstruction is/are reduced. The size and/or volume of the obstruction can be monitored using imaging (e.g., ultrasound, MRI, and/or other imaging).

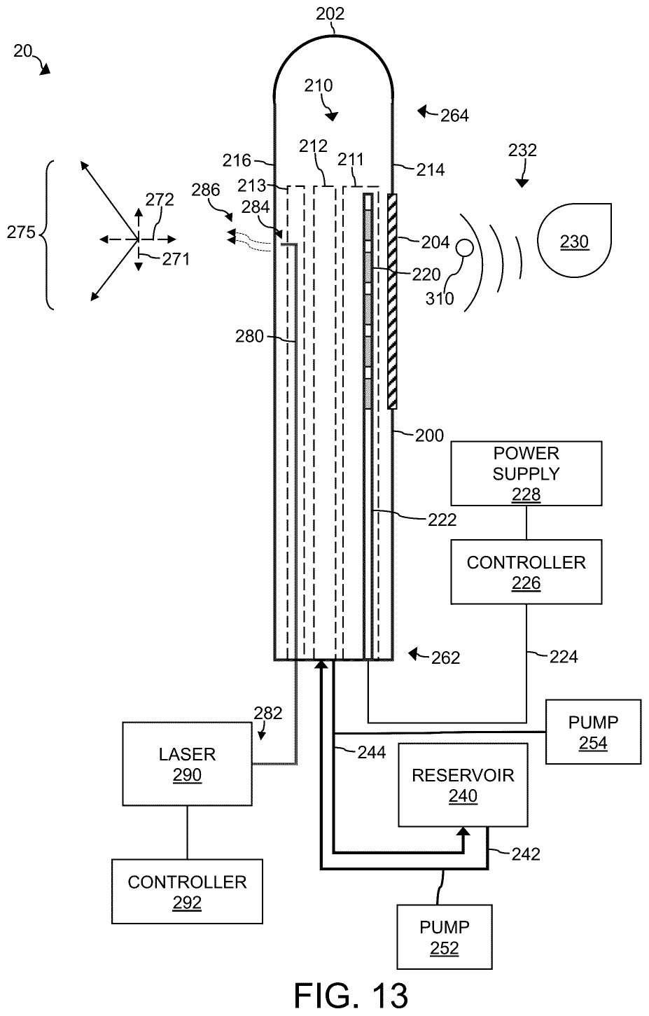

In some embodiments, after the size and/or volume of the obstruction is/are reduced in step 1003 and/or prior to step 1004, the ultrasound applicator 20 can be rotated (e.g., by 180) to align the ultrasound energy 232 with the target volume 230, for example as shown in FIG. 13 where the ultrasound applicator 20 is rotated by 180 degrees compared to FIGS. 11 and 12.

In some embodiments, laser energy and ultrasound energy can be used simultaneously and/or serially (e.g., alternatingly) to thermally treat the target volume. This can include one or more cycles of alternatingly rotating the ultrasound applicator (e.g., by about 180 degrees) between a first treatment of ultrasound energy and a second treatment of laser energy for example using ultrasound applicator 20, 30, 40, 50, or 60. Alternatively, the ultrasound applicator does not need to rotated using ultrasound applicator 70 such that laser energy and ultrasound energy can be applied simultaneously and/or serially while maintaining the same rotational orientation.

The laser energy can increase the rate and/or efficiency of treating a target volume. For example, when the target volume is obstructed by a calcification or another object, at least some (or all) ultrasound energy cannot reach the target volume. The laser energy can be used to break apart the calcification (or other obstruction) allowing effective and/or more efficient use of ultrasound energy on the target volume. Additionally or alternatively, the laser energy can be used to ablate the target volume in addition to or instead of the ultrasound energy.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. An ultrasound applicator comprising:
a shaft having a proximal end, a tip, and a length measured between the proximal end and the tip with respect to an axis;
a plurality of channels defined in the shaft and extending from the proximal end of the shaft along at least a portion of the length of the shaft, the plurality of channels including an ultrasound channel and an optical-energy channel;
one or more ultrasound transducers disposed in the ultrasound channel, the ultrasound transducer(s) configured to produce ultrasound energy in a first direction; and
one or more optical fibers disposed in the optical-energy channel, the optical fiber(s) having a first end configured to be optically coupled to a laser source and a second end configured to direct laser energy at a predetermined angle relative to the first direction.

2. The ultrasound applicator of claim 1, wherein the predetermined angle has a range of about 120 degrees to about 240 degrees whereby the ultrasound energy is directed towards a first side of the shaft and the laser energy is directed towards a second side of the shaft.

3. The ultrasound applicator of claim 2, wherein the predetermined angle is about 180 degrees.

4. The ultrasound applicator of claim 1, further comprising an optical window defined in the shaft, the optical window aligned with the second end of the optical fiber(s) such that the laser energy passes through the optical window, the optical window optically transparent to one or more wavelengths of the laser energy.

5. The ultrasound applicator of claim 1, wherein:
the predetermined angle is a first predetermined angle, and
the ultrasound applicator further comprises a mirror configured to reflect the laser energy at a second predetermined angle relative to the first direction.

6. The ultrasound applicator of claim 5, wherein the mirror is disposed in the optical-energy channel.

7. The ultrasound applicator of claim 5, wherein the mirror is electromechanically actuated such that the second predetermined angle is variable.

8. The ultrasound applicator of claim 1, wherein the predetermined angle has a range of about +60 degrees to about −60 degrees whereby the ultrasound energy and the laser energy are directed towards a same side of the shaft.

9. The ultrasound applicator of claim 1, further comprising a laser disposed in the optical-energy channel and optically coupled to the first end of the optical fiber(s).

10. A medical device comprising:
the ultrasound applicator of claim 1;
a laser optically coupled to the first end of the optical fiber(s); and
a power supply electrically coupled to the ultrasound transducer(s).

11. An ultrasound applicator comprising:
a shaft having a proximal end, a tip, and a length measured between the proximal end and the tip with respect to an axis;
a plurality of channels defined in the shaft and extending from the proximal end of the shaft along at least a portion of the length of the shaft, the plurality of channels including an ultrasound channel and an optical-energy channel;
one or more ultrasound transducers disposed in the ultrasound channel, the ultrasound transducer(s) configured to produce ultrasound energy in a first direction; and
a laser disposed in the optical-energy channel, the laser configured to direct laser energy at a predetermined angle relative to the first direction.

12. The ultrasound applicator of claim 11, wherein the predetermined angle has a range of about 120 degrees to about 240 degrees whereby the ultrasound energy is directed towards a first side of the shaft and the laser energy is directed towards a second side of the shaft.

13. The ultrasound applicator of claim 11, further comprising an optical window defined in the shaft, the optical window aligned with the laser such that the laser energy passes through the optical window, the optical window optically transparent to one or more wavelengths of the laser energy.

14. The ultrasound applicator of claim 11, wherein:
the predetermined angle is a first predetermined angle, and
the ultrasound applicator further comprises a mirror configured to reflect the laser energy at a second predetermined angle relative to the first direction.

15. The ultrasound applicator of claim 11, wherein the predetermined angle has a range of about +60 degrees to about −60 degrees whereby the ultrasound energy and the laser energy are directed towards a same side of the shaft.

16. A method for performing thermal therapy, comprising:
positioning an ultrasound applicator relative to a target volume in a mammal;
directing laser energy towards an obstruction located between the ultrasound applicator and the target volume, the laser energy emitted from one or more optical fibers and/or a laser disposed in an optical-energy channel defined in a shaft of the ultrasound applicator;
reducing a size of the obstruction; and
after the size of the obstruction is reduced, directing ultrasound energy towards the target volume, the ultrasound energy produced by one or more ultrasound transducers disposed in an ultrasound channel defined in the shaft of the ultrasound applicator.

17. The method of claim 16, wherein the obstruction comprises a calcification.

18. The method of claim 16, wherein:
the laser energy is directed towards a first side of the shaft,
the ultrasound energy is directed towards a second side of the shaft, and
the method further comprises after the size of the obstruction is reduced, rotating the shaft to align the ultrasound transducer(s) with the target volume.

19. The method of claim 16, wherein:
the optical fiber(s) and/or the laser is/are configured to direct the laser energy in a first direction, and
the method further comprises reflecting the laser energy, with a mirror, in a second direction towards the obstruction.

20. The method of claim 19, further comprising electromechanically pivoting the mirror to adjust the second direction.
